SEQUENCE LISTING (44 sequences)

SEQ ID NO: 1 - protein, 825 aa, Bifidobacterium adolescentis
MTSPVIGTPWKKLNAPVSEEAIEGVDKYWRAANYLSIGQIYLRSNPLMKEPFTREDVKHRLVGHWGTTPGLNFLIGHINRLIADHQQNTVIIMGPGHGGPAGTAQSYLDGTYTEYFPNITKDEAGLQKFFRQFSYPGGIPSHYAPETPGSIHEGGELGYALSHAYGAVMNNPSLFVPAIVGDGEAETGPLATGWQSNKLINPRTDGIVLPILHLNGYKIANPTILSRISDEELHEFFHGMGYEPYEFVAGFDNEDHLSIHRRFAELFETVFDEICDIKAAAQTDDMTRPFYPMIIFRTPKGWTCPKFIDGKKTEGSWRSHQVPLASARDTEAHFEVLKNWLESYKPEELFDENGAVKPEVTAFMPTGELRIGENPNANGGRIREELKLPKLEDYEVKEVAEYGHGWGQLEATRRLGVYTRDIIKNNPDSFRIFGPDETASNRLQAAYDVTNKQWDAGYLSAQVDEHMAVTGQVTEQLSEHQMEGFLEGYLLTGRHGIWSSYESFVHVIDSMLNQHAKWLEATVREIPWRKPISSMNLLVSSHVWRQDHNGFSHQDPGVTSVLLNKCFNNDHVIGIYFPVDSNMLLAVAEKCYKSTNKINAIIAGKQPAATWLTLDEARAELEKGAAEWKWASNVKSNDEAQIVLAATGDVPTQEIMAAADKLDAMGIKFKVVNVVDLVKLQSAKENNEALSDEEFAELFTEDKPVLFAYHSYARDVRGLIYDRPNHDNFNVHGYEEQGSTTTPYDMVRVNNIDRYELQAEALRMIDADKYADKINELEAFRQEAFQFAVDNGYDHPDYTDWVYSGVNTNKQGAISATAATAGDNE

SEQ ID NO: 2 - protein, 825 aa, Bifidobacterium breve
MTSPVIGTPWKKLNAPVSEESLEGVDKYWRVANYLSIGQIYLRSNPLMKAPFTREDVKHRLVGHWGTTPGLNFLIGHINRFIADHGQNTVIIMGPGHGGPAGTSQSYLDGTYTETFPKITKDEAGLQKFFRQFSYPGGIPSHFAPETPGSIHEGGELGYALSHAYGAIMDNPSLFVPAIVGDGEAETGPLATGWQSNKLVNPRTDGIVLPILHLNGYKIANPTILSRISDEELHEFFHGMGYEPYEFVAGFDDEDHMSIHRRFAELWETIWDEICDIKAAAQTDNVHRPFYPMLIFRTPKGWTCPKYIDGKKTEGSWRAHQVPLASARDTEAHFEVLKNWLESYKPEELFDANGAVKDDVLAFMPKGELRIGANPNANGGVIRDDLKLPNLEDYEVKEVAEYGHGWGQLEATRTLGAYTRDIIRNNPRDFRIFGPDETASNRLQASYEVTNKQWDAGYISDEVDEHMHVSGQVVEQLSEHQMEGFLEAYLLTGRHGIWSSYESFVHVIDSMLNQHAKWLEATVREIPWRKPIASMNLLVSSHVWRQDHNGFSHQDPGVTSVLLNKCFHNDHVIGIYFATDANMLLAIAEKCYKSTNKINAIIAGKQPAATWLTLDEARAELAKGAAAWDWASTAKNNDEAEVVLAAAGDVPTQEIMAASDKLKELGVKFKVVNVADLLSLQSAKENDEALSDEEFADIFTADKPVLFAYHSYAHDVRGLIYDRPNHDNFNVHGYEEEGSTTTPYDMVRVNRIDRYELTAEALRMIDADKYADKIDELEKFRDEAFQFAVDKGYDHPDYTDWVYSGVNTDKKGAVTATAATAGDNE

SEQ ID NO: 3 - DNA, 2478 bp, Artificial Sequence
  It is synthesized
atgacctctccagttattggtactccatggaaaaaattgaacgccccagtttctgaagaggctattgaaggtgttgataagtattggagagctgccaactatttgtccattggtcaaatctacttgaggtctaacccattgatgaaggaaccattcactagggaagatgttaagcacagattggttggtcattggggtactactccaggtttgaattttttgatcggtcacatcaacagattgatcgctgatcatcaacaaaacaccgttattatcatgggtccaggtcatggtggtccagctggtactgctcaatcttatttggatggtacttacaccgaatacttcccaaacattactaaggatgaagctggcttgcaaaagttcttcagacaattttcttacccaggtggtatcccatctcattatgctccagaaacaccaggttctattcatgaaggtggtgaattgggttatgctttgtctcatgcttatggtgctgttatgaacaacccatctttgtttgttccagctatagttggtgatggtgaagctgaaactggtccattggctactggttggcaatctaacaaattgattaacccaagaaccgacggtatcgttttgccaattttacatttgaacggttacaagatcgctaacccaaccattttgtccagaatctctgatgaagaattgcacgaattcttccatggtatgggttatgaaccatacgaatttgttgctggtttcgacaacgaagatcatttgtctatccatagaagattcgccgagttgttcgaaactgttttcgacgaaatttgcgatattaaggctgctgctcaaactgatgatatgactagaccattttaccccatgatcattttcagaactccaaaaggttggacctgtccaaagtttatcgatggtaaaaagactgaaggctcttggagatctcatcaagttccattggcatctgctagagatactgaagctcatttcgaagttttgaagaactggttggaaagctacaaacctgaagagttgtttgacgaaaatggtgcagttaagcctgaagttactgcttttatgccaacaggtgaattgagaattggtgaaaatccaaatgctaacggtggtagaatcagggaagaattgaaattgccaaagttggaagattacgaggtcaaagaagttgctgaatacggtcatggttggggtcaattggaagctactagaagattgggtgtttacaccagagacatcatcaagaacaatccagactccttcagaatttttggtccagacgaaactgcttctaacagattgcaagctgcttacgatgttaccaacaaacaatgggatgctggttatttgtctgcccaagttgatgaacatatggctgttactggtcaagttaccgaacaattgtcagaacatcaaatggaaggtttcttggagggttacttgttgactggtagacatggtatttggtcctcttacgaatctttcgttcacgttatcgactctatgttgaatcaacacgctaagtggttagaagctaccgttagagaaattccttggagaaagccaatctcctctatgaacttgttggtttcttcacacgtttggagacaagatcataacggtttttcacatcaagatccaggtgttacttctgtcttgttgaacaagtgtttcaacaacgatcatgtcatcggtatctacttcccagttgactctaatatgttgttggctgttgctgaaaagtgctacaaatctaccaacaagatcaacgctattatcgctggtaagcaaccagctgctacttggttgactttagatgaagctagagctgaattggaaaaaggtgctgctgaatggaaatgggcttctaatgttaagtccaacgatgaagctcaaatcgttttggctgctacaggtgatgttccaactcaagaaattatggctgcagctgataagttggatgctatgggtattaagttcaaggttgttaacgttgtcgacctggttaagttgcaatcagctaaagaaaacaacgaagccttgtccgacgaagaatttgctgagttgtttactgaagataagccagttttgttcgcctaccattcttacgctagagatgttagaggtttgatatacgatagaccaaaccacgataacttcaacgttcatggttatgaagaacaaggttctactactaccccatacgatatggttagagttaacaacatcgacaggtacgaattacaagctgaagccttgagaatgattgatgcagataagtacgccgacaagattaacgaactagaagcttttagacaagaggcctttcaattcgctgttgataatggttacgatcacccagattataccgattgggtttattctggtgtcaacactaacaagcaaggtgctatttctgctactgctgcaactgctggtgataatgaataa

SEQ ID NO: 4 - DNA, 2475 bp, Artificial Sequence
  It is synthesized
atgacctctccagttattggtactccatggaaaaaattgaacgccccagtttctgaagaatccttggaaggtgttgataagtattggagagttgccaactacttgtccattggtcaaatctacttgaggtctaacccattgatgaaggctccattcactagagaagatgttaagcacagattggttggtcattggggtactactccaggtttgaattttttgatcggtcacatcaacagattcattgctgatcatggtcaaaacaccgttattatcatgggtccaggtcatggtggtccagctggtacttctcaatcttatttggatggtacttacaccgagactttcccaaagattacaaaagatgaagctggcttgcagaagttcttcagacaattttcttatccaggtggtatcccatctcattttgctccagaaacaccaggttctattcatgaaggtggtgaattgggttatgctttgtctcatgcttatggtgccattatggataacccatctttgtttgttccagccatagttggtgatggtgaagctgaaactggtccattggctactggttggcaatctaacaaattggttaacccaagaaccgatggtatcgttttgccaatcttgcatttgaacggttacaagattgctaacccaaccatcttgtccagaatctctgatgaagaattgcatgagttcttccatggtatgggttatgaaccatacgaatttgttgctggtttcgatgatgaagatcacatgtccattcatagaagattcgctgaattgtgggaaaccatttgggacgaaatttgcgatattaaggctgctgctcaaaccgataatgttcatagaccattttaccccatgctgattttcagaactccaaaaggttggacttgcccaaagtacattgatggtaaaaagactgaaggttcttggagagcacatcaagttccattggcatctgctagagatactgaagctcatttcgaagttttgaagaactggttggaaagctacaaacctgaagagttgtttgatgctaatggtgctgttaaggatgatgttttggcttttatgccaaagggtgaattgagaattggtgctaatccaaatgcaaacggtggtgttattagagatgatctgaagttgccaaacttggaagattacgaagtcaaagaagttgccgaatacggtcatggttggggtcaattggaagctactagaactttgggtgcttacaccagagatatcattagaaacaacccaagagacttcagaattttcggtccagacgaaactgcttctaatagattgcaagcttcttacgaggttaccaacaaacaatgggatgctggttacatttccgatgaagttgatgaacatatgcacgtttctggtcaagttgtcgaacaattgtcagaacatcaaatggaaggttttttggaggcttacttgttgactggtagacatggtatttggtcctcttacgaatctttcgttcacgttatcgactctatgttgaatcaacacgctaaatggttggaagccaccgttagagaaattccttggagaaaacctattgcctccatgaacttgttggtttcttcacatgtttggagacaagatcacaacggtttctctcatcaagatccaggtgttacttctgtcttgttgaacaagtgtttccacaacgatcatgtcatcggtatctactttgctactgatgctaacatgttgttggctattgctgaaaagtgttacaagtccaccaacaagattaacgctattattgctggtaaacaaccagctgctacttggttgactttggatgaagctagagctgaattggctaaaggtgctgctgcttgggattgggcttctactgctaaaaacaatgatgaagccgaagttgttttagctgctgctggtgatgttccaactcaagaaattatggctgcttccgacaagttgaaagaattgggtgttaagttcaaggttgttaacgttgccgatttgttgtcattgcaatccgctaaagaaaacgacgaagctttgtcagacgaagaatttgctgatatcttcactgctgataagccagttttgttcgcttatcattcttacgcccatgatgtcagaggtttgatatacgatagaccaaaccacgataacttcaacgttcatggttatgaagaagaaggttctactactaccccatacgatatggttagagttaacagaatcgacaggtacgaattgactgctgaagctttgagaatgattgatgcagataagtacgccgataagatcgacgaattggaaaagttcagagatgaagcctttcaattcgcagttgataagggttatgatcacccagattataccgattgggtttactctggtgttaataccgataagaaaggtgcagttactgctactgctgcaacagctggtgataatgaa

SEQ ID NO: 5 - DNA, 50 bp, Artificial Sequence
  It is synthesized
atatttccgcattcatccttcaattgtgtttaaagattacggatatttaa

SEQ ID NO: 6 - DNA, 47 bp, Artificial Sequence
  It is synthesized
accaataactggagaggtcatttttagtttatgtatgtgttttttgt

SEQ ID NO: 7 - DNA, 50 bp, Artificial Sequence
  It is synthesized
gctatacgaagttattaggtgatatatgttatatagtctttttatttatg

SEQ ID NO: 8 - DNA, 20 bp, Artificial Sequence
  It is synthesized
ctatgttcactctggcgtgc

SEQ ID NO: 9 - DNA, 50 bp, Artificial Sequence; identical to SEQ ID NO: 5
  It is synthesized

SEQ ID NO: 10 - DNA, 47 bp, Artificial Sequence; identical to SEQ ID NO: 6
  It is synthesized

SEQ ID NO: 11 - DNA, 50 bp, Artificial Sequence
  It is synthesized
tgcaactgctggtgataatgaataagtctgaagaatgaatgatttgatga

SEQ ID NO: 12 - DNA, 46 bp, Artificial Sequence
  It is synthesized
cattatacgaagttatattaagggttattcgaactgcccattcagc

SEQ ID NO: 13 - DNA, 47 bp, Artificial Sequence
  It is synthesized
acaaaaaacacatacataaactaaaaatgacctctccagttattggt

SEQ ID NO: 14 - DNA, 50 bp, Artificial Sequence
  It is synthesized
tcatcaaatcattcattcttcagacttattcattatcaccagcagttgca

SEQ ID NO: 15 - DNA, 47 bp, Artificial Sequence; identical to SEQ ID NO: 13
  It is synthesized

SEQ ID NO: 16 - DNA, 50 bp, Artificial Sequence
  It is synthesized
tcatcaaatcattcattcttcagacttattcattatcaccagctgttgca

SEQ ID NO: 17 - DNA, 46 bp, Artificial Sequence
  It is synthesized
gctgaatgggcagttcgaataacccttaatataacttcgtataatg

SEQ ID NO: 18 - DNA, 50 bp, Artificial Sequence
  It is synthesized
cataaataaaaagactatataacatatatcacctaataacttcgtatagc

SEQ ID NO: 19 - DNA, 24 bp, Artificial Sequence
  It is synthesized
ctccgtttatgataagatgatggc

SEQ ID NO: 20 - DNA, 21 bp, Artificial Sequence
  It is synthesized
gttaattgcctccgcatctaa

SEQ ID NO: 21 - protein, 520 aa, Escherichia coli
MKPEDFRASTQRPFTGEEYLKSLQDGREIYIYGERVKDVTTHPAFRNAAASVAQLYDALHKPEMQDSLCWNTDTGSGGYTHKFFRVAKSADDLRHERDAIAEWSRLSYGWMGRTPDYKAAFGCALGGTPGFYGQFEQNARNWYTRIQETGLYFNHAIVNPPIDRHLPTDKVKDVYIKLEKETDAGIIVSGAKVVATNSALTHYNMIGFGSAQVMGENPDFALMFVAPMDADGVKLISRASYEMVAGATGSPYDYPLSSRFDENDAILVMDNVLIPWENVLLYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQSSGNMDKMMAMVDRCLSEYDQNGWTVPHLHNNDDINMLDKLLK

SEQ ID NO: 22 - protein, 170 aa, Escherichia coli
MQLDEQRLRFRDAMASLSAAVNIITTEGDAGQCGITATAVCSVTDTPPSLMVCINANSAMNPVFQGNGKLCVNVLNHEQELMARHFAGMTGMAMEERFSLSCWQKGPLAQPVLKGSLASLEGEIRDVQAIGTHLVYLVEIKNIILSAEGHGLIYFKRRFHPVMLEMEAAI

SEQ ID NO: 23 - DNA, 1563 bp, Artificial Sequence
  It is synthesized
atgaaaccagaagatttccgcgccagtacccaacgtccgttcaccggggaagagtatctgaaaagcctgcaggatggtcgcgagatctatatctatggcgagcgagtgaaagacgtcactactcatccggcatttcgtaatgcggctgcgtctgttgcccaactgtacgacgcgctacacaaaccggagatgcaggactctctgtgctggaacaccgacaccggcagcggcggctatacccataaattcttccgcgtggcgaaaagtgccgacgacctgcgccacgaacgcgatgccatcgctgagtggtcacgcctgagctatggctggatgggccgtaccccagactacaaagctgctttcggttgcgcactgggcggaactccgggcttttacggtcagttcgagcagaacgcccgtaactggtacacccgtattcaggaaactggcctctactttaaccacgcgattgttaacccaccgatcgatcgtcatttgccgaccgataaagtaaaagacgtttacatcaagctggaaaaagagactgacgccgggattatcgtcagcggtgcgaaagtggttgccaccaactcggcgctgactcactacaacatgattggcttcggctcggcacaagtaatgggcgaaaacccggacttcgcactgatgttcgttgcgccaatggatgccgatggcgtcaaattaatctcccgcgcctcttatgagatggtcgcgggtgctaccggctcaccgtatgactacccgctctccagccgcttcgatgagaacgatgcgattctggtgatggataacgtgctgatcccatgggaaaacgtgctgctctaccgcgattttgatcgctgccgtcgctggacgatggaaggcggtttcgcccgtatgtatccgctgcaagcctgtgtgcgcctggcagtgaaactcgacttcattacggcactgctgaaaaaatcactcgaatgtaccggcaccctggagttccgtggtgtgcaggccgatctcggtgaagtggtggcgtggcgcaacaccttctgggcattgagtgactcgatgtgttctgaagcgacgccgtgggtcaacggggcttatttaccggatcatgccgcactgcaaacctatcgcgtactggcaccaatggcctacgcgaagatcaaaaacattatcgaacgcaacgttaccagtggcctgatctatctcccttccagtgcccgtgacctgaacaatccgcagatcgaccagtatctggcgaagtatgtgcgcggttcgaacggtatggatcacgtccagcgcatcaagatcctcaaactgatgtgggatgccattggcagcgagtttggtggtcgtcacgaactgtatgaaatcaactactccggtagccaggatgagattcgcctgcagtgtctgcgccaggcacaaagctccggcaatatggacaagatgatggcgatggttgatcgctgcctgtcggaatacgaccagaacggctggactgtgccgcacctgcacaacaacgacgatatcaacatgctggataagctgctgaaataa

SEQ ID NO: 24 - DNA, 513 bp, Artificial Sequence
  It is synthesized
atgcaattagatgaacaacgcctgcgctttcgtgacgcaatggccagcctgtcggcagcggtaaatattatcaccaccgagggcgacgccggacaatgcgggattacggcaacggccgtctgctcggtcacggatacaccaccatcgctgatggtgtgcattaacgccaacagtgcgatgaacccggtttttcagggcaacggtaagttgtgcgtcaacgtcctcaaccatgagcaggaactgatggcacgccacttcgcgggcatgacaggcatggcgatggaagagcgttttagcctctcatgctggcaaaaaggtccgctggcgcagccggtgctaaaaggttcgctggccagtcttgaaggtgagatccgcgatgtgcaggcaattggcacacatctggtgtatctggtggagattaaaaacatcatcctcagtgcagaaggtcacggacttatctactttaaacgccgtttccatccggtgatgctggaaatggaagctgcgatttaa

SEQ ID NO: 25 - DNA, 22 bp, Artificial Sequence
  It is synthesized
atgaaaccagaagatttccgcg

SEQ ID NO: 26 - DNA, 22 bp, Artificial Sequence
  It is synthesized
ttatttcagcagcttatccagc

SEQ ID NO: 27 - DNA, 23 bp, Artificial Sequence
  It is synthesized
atgcaattagatgaacaacgcct

SEQ ID NO: 28 - DNA, 23 bp, Artificial Sequence
  It is synthesized
ttaaatcgcagcttccatttcca

SEQ ID NO: 29 - DNA, 7347 bp, Artificial Sequence
  It is synthesized
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacatgaaaccagaagatttccgcgccagtacccaacgtccgttcaccggggaagagtatctgaaaagcctgcaggatggtcgcgagatctatatctatggcgagcgagtgaaagacgtcactactcatccggcatttcgtaatgcggctgcgtctgttgcccaactgtacgacgcgctacacaaaccggagatgcaggactctctgtgctggaacaccgacaccggcagcggcggctatacccataaattcttccgcgtggcgaaaagtgccgacgacctgcgccacgaacgcgatgccatcgctgagtggtcacgcctgagctatggctggatgggccgtaccccagactacaaagctgctttcggttgcgcactgggcggaactccgggcttttacggtcagttcgagcagaacgcccgtaactggtacacccgtattcaggaaactggcctctactttaaccacgcgattgttaacccaccgatcgatcgtcatttgccgaccgataaagtaaaagacgtttacatcaagctggaaaaagagactgacgccgggattatcgtcagcggtgcgaaagtggttgccaccaactcggcgctgactcactacaacatgattggcttcggctcggcacaagtaatgggcgaaaacccggacttcgcactgatgttcgttgcgccaatggatgccgatggcgtcaaattaatctcccgcgcctcttatgagatggtcgcgggtgctaccggctcaccgtatgactacccgctctccagccgcttcgatgagaacgatgcgattctggtgatggataacgtgctgatcccatgggaaaacgtgctgctctaccgcgattttgatcgctgccgtcgctggacgatggaaggcggtttcgcccgtatgtatccgctgcaagcctgtgtgcgcctggcagtgaaactcgacttcattacggcactgctgaaaaaatcactcgaatgtaccggcaccctggagttccgtggtgtgcaggccgatctcggtgaagtggtggcgtggcgcaacaccttctgggcattgagtgactcgatgtgttctgaagcgacgccgtgggtcaacggggcttatttaccggatcatgccgcactgcaaacctatcgcgtactggcaccaatggcctacgcgaagatcaaaaacattatcgaacgcaacgttaccagtggcctgatctatctcccttccagtgcccgtgacctgaacaatccgcagatcgaccagtatctggcgaagtatgtgcgcggttcgaacggtatggatcacgtccagcgcatcaagatcctcaaactgatgtgggatgccattggcagcgagtttggtggtcgtcacgaactgtatgaaatcaactactccggtagccaggatgagattcgcctgcagtgtctgcgccaggcacaaagctccggcaatatggacaagatgatggcgatggttgatcgctgcctgtcggaatacgaccagaacggctggactgtgccgcacctgcacaacaacgacgatatcaacatgctggataagctgctgaaataacgcagcaggaggttaagatgcaattagatgaacaacgcctgcgctttcgtgacgcaatggccagcctgtcggcagcggtaaatattatcaccaccgagggcgacgccggacaatgcgggattacggcaacggccgtctgctcggtcacggatacaccaccatcgctgatggtgtgcattaacgccaacagtgcgatgaacccggtttttcagggcaacggtaagttgtgcgtcaacgtcctcaaccatgagcaggaactgatggcacgccacttcgcgggcatgacaggcatggcgatggaagagcgttttagcctctcatgctggcaaaaaggtccgctggcgcagccggtgctaaaaggttcgctggccagtcttgaaggtgagatccgcgatgtgcaggcaattggcacacatctggtgtatctggtggagattaaaaacatcatcctcagtgcagaaggtcacggacttatctactttaaacgccgtttccatccggtgatgctggaaatggaagctgcgattcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 30 - protein, 824 aa, Bifidobacterium dentium
TSPVIGTPTLLLAAPVSGGAIGGVALTTAAAATLSIGGITLASAPLMLGPPTAGAVLHALVGHTGTTPGLAPLIGHIAALIAAHGGATVIIMGPGHGGPAGTAGSTLAGTTTGTPPAITLAGAGLGLPPAGPSTPGGIPSHTAPGTPGSIHGGGGLGTALSHATGAVMAAPSLPVPAIVGAGGAGTGPLATGTGSALLVAPATAGIVLPILHLAGTLIAAPTILSAISAGGLHGPPHGMGTGPTGPVAGPAAGAHLSIHAAPAGLPGTVPAGICAILAAAGTAAMTAPPTPMIIPATPLGTTCPLPIAGLLTGGSTASHGVPLASAAATGAHPGVLLATLGSTLPGGLPAAAGAVLPGVTAPMPTGGLAIGGAPAAAGGAIAGGLALPALGATGVLGVAGTGHGTGGLGATAALGVTTAAIILAAPASPAIPGPAGTASAALGAATAVTALGTAAGTLSAGVAGHMAVTGGVTGGLSGHGMGGPLGATLLTGAHGITSSTGSPVHVIASMLAGHALTLGATVAGIPTALPISSMALLVSSHVTAGAHAGPSHGAPGVTSVLLALCPAAAHVIGITPPVASAMLLAVAGLCTLSTAMIAAIIAGLGPAATTLTLAGAAAGLGLGAAGTGTASTALSAAGAGIVLASAGAVPAGGIMAAAALLAAMGILPLVVAVVALVLLGSTLGAAGAISAAAPAALPTGALPVLPATHSTAAAVAGLITAAPAHAAPAVHGTGGGGSTTTPTAMVAVAAIAATGLVAGALAMIAAALTAALIAGLGAPALGAPGPAVAAGTAHPATTATVTSGVATALGGAVSATAATAGAAG

SEQ ID NO: 31 - DNA, 2478 bp, Artificial Sequence
  It is synthesized
atgacctctccagttattggtactccatggaaaaaattgaacgccccagtttctgaagaggctattgaaggtgttgataagtattggagagctgccaactatttgtccattggtcaaatctacttgaggtctaacccattgatgaaggaaccattcactagggaagatgttaagcacagattggttggtcattggggtactactccaggtttgaattttttgatcggtcacatcaacagattgatcgctgatcatcaacaaaacaccgttattatcatgggtccaggtcatggtggtccagctggtactgctcaatcttatttggatggtacttacaccgaatacttcccaaacattactaaggatgaagctggcttgcaaaagttcttcagacaattttcttacccaggtggtatcccatctcattatgctccagaaacaccaggttctattcatgaaggtggtgaattgggttatgctttgtctcatgcttatggtgctgttatgaacaacccatctttgtttgttccagctatagttggtgatggtgaagctgaaactggtccattggctactggttggcaatctaacaaattggttaacccaagaaccgatggtatcgttttgccaatcttgcatttgaacggttacaagattgctaacccaaccatcttgtccagaatctctgatgaagaattgcatgagttcttccatggtatgggttatgaaccatacgaatttgttgctggtttcgacaacgaagatcatttgtctatccatagaagattcgccgagttgttcgaaactgttttcgacgaaatttgcgatattaaggctgctgctcaaactgatgatatgactagaccattttaccccatgatcattttcagaactccaaaaggttggacctgtccaaagtttatcgatggtaaaaagactgaaggttcttggagatcacatcaagttccattggcatctgctagagatactgaagctcatttcgaagttttgaagaactggttggaaagctacaaacctgaagagttgtttgatgctaatggtgcagtaaagcctgaagttactgcttttatgccaacaggtgaattgagaattggtgaaaatccaaatgctaacggtggtagaatcagggaagaattgaatttgccagctttggaagattacgaagtcaaagaagttgctgaatacggtcatggttggggtcaattggaagctactagaagattgggtgtttacaccagagatatcatcaagaacaatccagactccttcagaatttttggtccagacgaaactgcttctaacagattgcaagctgcttacgatgttaccaacaaacaatgggatgctggttatttgtctgcccaagttgatgaacatatggctgttactggtcaagttaccgaacaattgtcagaacatcaaatggaaggttttttggaggcttacttgttgactggtagacatggtatttggtcctcttacgaatctttcgttcacgttatcgactctatgttgaatcaacacgctaaatggttggaagccactgttagagaaattccttggagaaagccaatctcctctatgaacttgttggtttcttcacacgtttggagacaagatcataacggtttctctcatcaagatccaggtgttacttccgtcttgttgaacaagtgtttcaacaacgatcatgtcatcggtatctacttcccagttgactctaatatgttgttggctgttgctgaaaagtgctacaagtctaccgatatgattaacgctattatcgctggtaagcaaccagctgctacttggttgactttagatgaagctagagctgaattggaaaaaggtgctgctgaatgggaatgggcttctactgctaaatcaaatgatgaagcccaaatcgttttggcttctgctggtgatgttccagcacaagaaattatggctgcagctgataagttggatgctatgggtattaagttcaaggttgttaacgttgtcgacctggttaagttgcaatctaccaaagaaaacgatgaagccatttccgatgcagattttgctgatttgttcaccgaagataagccagttttgttcgcttatcattcctacgctagagatgtcagaggtttgatatacgatagaccaaaccacgataacttcaacgttcatggttatgaagaacaaggttctactactaccccatacgatatggttagagttaacaacatcgacaggtatgaattggttgctgaagccttgagaatgattgatgcagataagtacgctgacaagatcgatgaattagaggcttttagaaaagaggccttccaattcgctgttgataatggttacgatcatccagattacaccgattgggtttattctggtgttaataccaacaagcaaggtgctgtttctgctactgctgctacagctggtgataatgaataa

SEQ ID NO: 32 - DNA, 47 bp, Artificial Sequence; identical to SEQ ID NO: 13
  It is synthesized

SEQ ID NO: 33 - DNA, 50 bp, Artificial Sequence
  It is synthesized
tcatcaaatcattcattcttcagacttattcattatcaccagctgtagca

SEQ ID NO: 34 - protein, 825 aa, Bifidobacterium animalis
MTNPVIGTPWQKLDRPVSEEAIEGMDKYWRVANYMSIGQIYLRSNPLMKEPFTRDDVKHRLVGHWGTTPGLNFLLAHINRLIADHQQNTVFIMGPGHGGPAGTAQSYIDGTYTEYYPNITKDEAGLQKFFRQFSYPGGIPSHFAPETPGSIHEGGELGYALSHAYGAIMDNPSLFVPCIIGDGEAETGPLATGWQSNKLVNPRTDGIVLPILHLNGYKIANPTILARISDEELHDFFRGMGYHPYEFVAGFDNEDHLSIHRRFAELFETIFDEICDIKAAAQTDDMTRPFYPMLIFRTPKGWTCPKFIDGKKTEGSWRAHQVPLASARDTEAHFEVLKGWMESYKPEELFNADGSIKEDVTAFMPKGELRIGANPNANGGRIREDLKLPELDQYEITGVKEYGHGWGQVEAPRSLGAYCRDIIKNNPDSFRVFGPDETASNRLNATYEVTKKQWDNGYLSALVDENMAVTGQVVEQLSEHQCEGFLEAYLLTGRHGIWSSYESFVHVIDSMLNQHAKWLEATVREIPWRKPISSVNLLVSSHVWRQDHNGFSHQDPGVTSVLLNKTFNNDHVTNIYFATDANMLLAIAEKCFKSTNKINAIFAGKQPAATWITLDEARAELEAGAAEWKWASNAKSNDEVQVVLAAAGDVPTQEIMAASDALNKMGIKFKVVNVVDLIKLQSSKENDEAMSDEDFADLFTADKPVLFAYHSYAQDVRGLIYDRPNHDNFTVVGYKEQGSTTTPFDMVRVNDMDRYALQAKALELIDADKYADKINELNEFRKTAFQFAVDNGYDIPEFTDWVYPDVKVDETSMLSATAATAGDNE

SEQ ID NO: 35 - protein, 825 aa, Bifidobacterium bifidum
MTSPVIGTPWKKLNAPVSEEALEGVDKYWRVANYLSIGQIYLRSNPLMKEPFTREDVKHRLVGHWGTTPGLNFLIGHINRFIADHGQNTVFIMGPGHGGPAGTSQSYLDGTYTETYPNITKDEAGLQKFFRQFSYPGGIPSHFAPETPGSIHEGGELGYALSHAYGAIMDNPSLFVPCIVGDGEAETGPLATGWQSNKLVNPRTDGIVLPILHLNGYKIANPTILSRISDEELHEFFHGMGYEPYEFVAGFDDEDHMSIHRRFAELFESVWDEICDIKAAANTDNMHRPFYPMIIFRTPKGWTCPKYIDGKKTEAPGAPTRVPLASARDTEAHFEVLKNWLESYKPEELFDANGAVKDDVLAFMPKGELRIGANPNANGGVIRKDLVLPALEDYEVKEVKEFGHGWGQLEATRRLGVYTRDIIKNNMHDFRIFGPDETASNRLQASYEVTNKQWDAGYISDEVDEHMHVSGQVVEQLSEHQMEGFLEAYLLTGRHGIWSSYESFVHVIDSMLNQHAKWLEATVREIPWRKPIASMNLLVSSHVWRQDHNGFSHQDPGVTSVLLNKCFHNDHVIGIYFATDANMLLAIAEKCYKSTNKINAIIAGKQPAATWLTLDEARAELAKGAAAWDWASTAKTNDEAQVVLAAAGDVPTQEIMAASDKLKALGIKFKVVNVADLLSLQSAKENDEALTDEEFADIFTADKPVLFAYHSYAHDVRGLIYDRPNHDNFNVHGYEEEGSTTTPYDMVRVNELDRYELTAEALRMIDADKYADEIQKLEDFRQEAFQFAVDKGYDHPDYTDWVYSGVKTDKKGAVTATAATAGDNE

SEQ ID NO: 36 - protein, 825 aa, Bifidobacterium lactis; identical to SEQ ID NO: 34

SEQ ID NO: 37 - protein, 825 aa, Bifidobacterium longum
MTSPVIGTPWKKLNAPVSEEALEGVDKYWRVANYLSIGQIYLRSNPLMKEPFTREDVKHRLVGHWGTTPGLNFLIGHINRFIADHGQNTVIIMGPGHGGPAGTSQSYLDGTYTETFPKITKDEAGLQKFFRQFSYPGGIPSHFAPETPGSIHEGGELGYALSHAYGAIMDNPSLFVPAIVGDGEAETGPLATGWQSNKLVNPRTDGIVLPILHLNGYKIANPTILSRISDEELHEFFHGMGYEPYEFVAGFDDEDHMSIHRRFAELWETIWDEICDIKAAAQTDNVHRPFYPMLIFRTPKGWTCPKYIDGKKTEGSWRAHQVPLASARDTEAHFEVLKNWLESYKPEELFDANGAVKDDVLAFMPKGELRIGANPNANGGVIRDDLKLPNLEDYEVKEVAEYGHGWGQLEATRTLGVYTRDIIKNNPRDFRIFGPDETASNRLQASYEVTNKQWDAGYISDEVDEHMHVSGQVVEQLSEHQMEGFLEAYLLTGRHGIWSSYESFVHVIDSMLNQHAKWLEATVREIPWRKPIASMNLLVSSHVWRQDHNGFSHQDPGVTSVLLNKCFHNDHVIGIYFATDANMLLAIAEKCYKSTNKINAIIAGKQPAATWLTLDEARAELEKGAAAWDWASTAKNNDEAEVVLAAAGDVPTQEIMAASDKLKELGVKFKVVNVVDLLSLQSAKENDEALTDEEFADIFTADKPVLFAYHSYAHDVRGLIYDRPNHDNFNVHGYEEEGSTTTPYDMVRVNRIDRYELTAEALRMIDADKYADKIDELEKFRDEAFQFAVDKGYDHPDYTDWVYSGVNTDKKGAVTATAATAGDNE

SEQ ID NO: 38 - protein, 825 aa, Bifidobacterium magnum
MTSPVIGTPWTKLDEPVSEETLEGIDKYWRAANYLSIGQIYLRSNPLMKEPFTRDNVKHRLVGHWGTTPGLNFLLGHINHLIAAHKQNTVIIMGPGHGGPAGTAQSYLDGTYTEYYPKITKDEAGLQKFFRQFSYPGGIPSHFAPETPGSIHEGGELGYALSHAYGAVMNNPSLFVPAIVGDGEAETGPLATGWQSNKLVNPRTDGIVLPILHLNGYKIANPSILSRISDEELHDFFHGMGYEPYEFVAGFDNEDHLSIHRRFADLFESIFDEICDIKAQAQTDDMTRPFYPMIIFRTPKGWTCPKYIDGKKTEGSWRAHQVPLASARDTEAHFEVLKHWLESYKPEELFAADGSIKPEVTSFMPEGELRIGENPNANGGRIREDLKLPKLEDYEVNEVKEYGHGWGQLEATRRMGVYTRDVIKLNPDSFRIFGPDETASNRLAAAYEVTNKQWDAGYLSAQVDEHMAVTGQVTEQLSEHQMEGFLEGYLLTGRHGVWSSYESFVHVIDSMLNQHAKWLEATVREIPWRKPISSVNLLVSSHVWRQDHNGFSHQDPGVTSVLLNKTFNNDHVVNIYFPVDANMLLAVTERCYKSTNEINAIIAGKQPAATWLTLDEARAELDKGAAEWTWASNTDNNDDVQVVLACAGDVPTQEIMAAADKLSAFGIKFKVVNVVDLLKLQSAKENDEALSDAEFSDLFTADKPVLFAYHSYAHDVRGLIYDRPNHDNFNVHGYKEQGSTTTPYDMVRVNDMDRYALTAEALRMVDKDKYAADIDKLEAFRKEAFQFAVDKGYDHPDYTDWLWHGVKSDKAGAVTATAATAGDNE

SEQ ID NO: 39 - protein, 825 aa, Bifidobacterium pseudolongum
MTNPVIGTPWQKLDRPVSEEAIEGMDKYWRVTNYMSIGQIYLRSNPLMKEPFTRDDVKHRLVGHWGTTPGLNFLLAHINRLIADHQQNTVFIMGPGHGGPAGTSQSYVDGTYTEYYPNITKDEAGLQKFFRQFSYPGGIPSHFAPETPGSIHEGGELGYALSHAYGAVMNNPSLFVPCIIGDGEAETGPLATGWQSNKLVNPRTDGIVLPILHLNGYKIANPTILARISDEELHDFFRGMGYHPYEFVAGFDNEDHMSIHRRFAELFETIFDEICDIKLRPQTDDMTRPFYPMLIFRTPKGWTCPKFIDGKKTEGSWRAHQVPLASARDTEEHFEVLKGWMESYKPEELFNADGSIKDDVTAFMPKGELRIGANPNANGGVIREDLKLPELDQYEVTGVKEYGHGWGQVEAPRALGAYCRDIIKNNPDSFRIFGPDETASNRLNATYEVTDKQWDNGYLSGLVDEHMAVTGQVTEQLSEHQCEGFLEAYLLTGRHGIWSSYESFVHVIDSMLNQHAKWLEATVREIPWRKPISSVNLLVSSHVWRQDHNGFSHQDPGVTSLLINKTFNNDHVTNIYFATDTNMLLAISEKCFKSTNKINAIFAGKQPAPTWVTLDEARAELEAGAAEWKWASNAENNDEVQVVLASAGDVPTQELMAASDALNKMGIKFKVVNVVDLLKLQSRENNDEALTDEEFTELFTADKPVLFAYHSYAQDVRGLIYDRPNHDNFHVVGYKEQGSTTTPFDMVRVNDMDRYALQAAALKLIDADKYADKIDELNAFRKKAFQFAVDNGYDIPEFTDWVYPDVKVDETQMLSATAATAGDNE

SEQ ID NO: 40 - protein, 780 aa, Aspergillus nidulans
MPGEVIERPNPAPKPSHVPDLVEKLIIPAQKTKLEKSDCDALHKYRRAAAYIAAGHWGTCPGLILVYSHLNYLIKKQNLDMLYVVGPGHGAPGLLASLWLEGSLGKFYPQYTKDKEGLHNLISTFSTSAGLPSHINAETPGAIHEGGELGYALSVSFGAVMDNPDLIVTCVVGDGEAETGPTATSWHAIKYIDPAESGAVLPILHVNGFKISERTIFGCMDNREIVCLFTGYGYQVRIVEDLEDIDNDLHSAMSWAVEEIRNIQKAARSGKPIMKPQWPMIVLRTPKGWSGPKELHGQFIEGSFHSHQVPLPNAKKDDEELQALQKWLSSYKPDELFTESGDVIDEILSIIPSDDKKLGMRPEAYKTHLPPDLPDWRQFCVKKGDQFSAMKAIGSFIDQVFVKNPHTVRLFSPDELESNKLSAALSHTGRNFQWDEFSNAKGGRVIEVLSEHLCQGFMQGYTLTGRTGIFPSYESFLGIIHTMMVQYAKFAKMAKETAWHHDVSSINYIETSTWARQEHNGFSHQNPSFIGAVLKLKPYAARVYLPPDANTFLTTLHHCLKSKNYINLMVGSKQPTPVYLSPEEAESHCRAGASIFKFCSTDGGLRPDVVLVGIGVEVMFEVIKAAAILRERCPELRVRVVNVTDLFILENEGAHPHALKHEAFDNLFTEDRSIHFNYHGYVNELQGLLFGRPRLDRATIKGYKEEGSTTTPFDMMLVNEVSRYHVAKAAVTGGARFNEKVKLRHQELCSEFDHNIAETRKYIMNNHQDPEDTYNMPSFN

SEQ ID NO: 41 - protein, 796 aa, Clostridium acetobutylicum
MQSIIGKHKDEGKITPEYLKKIDAYWRAANFISVGQLYLLDNPLLREPLKPEHLKRKVVGHWGTIPGQNFIYAHLNRVIKKYDLDMIYVSGPGHGGQVMVSNSYLDGTYSEVYPNVSRDLNGLKKLCKQFSFPGGISSHMAPETPGSINEGGELGYSLAHSFGAVFDNPDLITACVVGDGEAETGPLATSWQANKFLNPVTDGAVLPILHLNGYKISNPTVLSRIPKDELEKFFEGNGWKPYFVEGEDPEAMHKLMAETLDIVTEEILNIQKNARENNDCSRPKWPMIVLRTPKGWTGPKFVDGVPNEGSFRAHQVPLAVDRYHTENLDQLEEWLKSYKPEELFDENYRLIPELEELTPKGNKRMAANLHANGGLLLRELRTPDFRDYAVDVPTPGSTVKQDMIELGKYVRDVVKLNEDTRNFRIFGPDETMSNRLWAVFEGTKRQWLSEIKEPNDEFLSNDGRIVDSMLSEHLCEGWLEGYLLTGRHGFFASYEAFLRIVDSMITQHGKWLKVTSQLPWRKDIASLNLIATSNVWQQDHNGYTHQDPGLLGHIVDKKPEIVRAYLPADANTLLAVFDKCLHTKHKINLLVTSKHPRQQWLTMDQAVKHVEQGISIWDWASNDKGQEPDVVIASCGDTPTLEALAAVTILHEHLPELKVRFVNVVDMMKLLPENEHPHGLSDKDYNALFTTDKPVIFAFHGFAHLINQLTYHRENRNLHVHGYMEEGTITTPFDMRVQNKLDRFNLVKDVVENLPQLGNRGAHLVQLMNDKLVEHNQYIREVGEDLPEITNWQWHV

SEQ ID NO: 42 - protein, 813 aa, Leuconostoc mesenteroides
MADFDSKEYLELVDKWWRATNYLSAGMIFLKSNPLFSVTNTPIKAEDVKVKPIGHWGTISGQTFLYAHANRLINKYGLNMFYVGGPGHGGQVMVTNAYLDGAYTEDYPEITQDIEGMSHLFKRFSFPGGIGSHMTAQTPGSLHEGGELGYSLSHAFGAVLDNPDQVAFAVVGDGEAETGPSMASWHSIKFLNAKNDGAVLPVLDLNGFKISNPTIFSRMSDEEITKFFEGLGYSPRFIENDDIHDYATYHQLAANILDQAIEDIQAIQNDARENGKYQDGEIPAWPVIIARLPKGWGGPTHDASNNPIENSFRAHQVPLPLEQHDLATLPEFEDWMNSYKPEELFNADGSLKDELKAIAPKGDKRMSANPITNGGADRSDLKLPNWREFANDINDDTRGKEFADSKRNMDMATLSNYLGAVSQLNPTRFRFFGPDETMSNRLWGLFNVTPRQWMEEIKEPQDQLLSPTGRIIDSQLSEHQAEGWLEGYTLTGRVGIFASYESFLRVVDTMVTQHFKWLRHASEQAWRNDYPSLNLIATSTAFQQDHNGYTHQDPGMLTHLAEKKSNFIREYLPADGNSLLAVQERAFSERHKVNLLIASKQPRQQWFTVEEAEVLANEGLKIIDWASTAPSSDVDITFASAGTEPTIETLAALWLINQAFPDVKFRYVNVVELLRLQKKSEPNMNDERELSAEEFNKYFQADTPVIFGFHAYENLIESFFFERKFTGDVYVHGYREDGDITTTYDMRVYSHLDRFHQAKEAAEILSANGKIDQAAADTFIAKMDDTLAKHFQVTRNEGRDIEEFTDWTWSPLK

SEQ ID NO: 43 - protein, 788 aa, Lactobacillus paraplantarum
MTTDYSSPAYLQKVDKYWRAANYLSVGQLYLKDNPLLQQPLKASDVKVHPIGHWGTIAGQNFIYAHLNRVINKYGLKMFYVEGPGHGGQVMVSNSYLDGTYTDIYPEITQDVEGMQKLFKQFSFPGGVASHAAPETPGSIHEGGELGYSISHGVGAILDNPDEIAAVVVGDGESETGPLATSWQSTKFINPINDGAVLPILNLNGFKISNPTIFGRTSDAKIKEYFESMNWEPIFVEGDDPEKVHPALAKAMDEAVEKIKAIQKHARENDDATLPVWPMIVFRAPKGWTGPKSWDGDKIEGSFRAHQIPIPVDQNDMEHADALVDWLESYQPKELFNEDGSLKDDIKEIIPTGDSRMAANPITNGGVDPKALNLPNFRDYAVDTSKEGANVKQDMLVWSDYLRDVIKKNPDNFRLFGPDETMSNRLYGVFETTNRQWMEDIHPDSDQYEAPAGRVLDAQLSEHQAEGWLEGYVLTGRHGLFASYEAFLRVVDSMLTQHFKWLRKANELDWRKKYPSLNIIAASTVFQQDHNGYTHQDPGALTHLAEKKPEYIREYLPADANTLLAVGDVIFRSQEKINYVVTSKHPRQQWFSIEEAKQLVDNGLGIIDWASTDQGSEPDIVFAAAGTEPTLETLAAIQLLHDSFPEMKIRFVNVVDILKLRSPEKDPRGLSDAEFDHYFTKDKPVVFAFHGYEDLVRDIFFDRHNHNLYVHGYRENGDITTPFDVRVMNQMDRFDLAKSAIAAQPAMENTGAAFVQSMDNMLAKHNAYIRDAGTDLPEVNDWQWKGLK

SEQ ID NO: 44 - protein, 796 aa, Lactobacillus plantarum
MSEAIKSKTVDYSSDEYLKRVDEYWRAANYISVGQLYLLNNPLLREPLKATDVKVHPIGHWGTIAGQNFIYAHLNRAINKYGLNMFYIEGPGHGGQVMVSNSYLDGTYTETYPKITQDKAGMKRLFKQFSFPGGVASHADPKTPGSIHEGGELGYSILHGAGAVLDNPGLIAATVVGDGESETGPLATSWQVNKFLNPITDGTVLPILNLNGFKISNPTVLSRESHEELEDYFKGLGWDPHFVEGTDPAKMHKIMAEELDKVIEEIHAIRKNAKDNNDESRPKWPMIVFRAPKGWTGPKSWDGEPIEGSFRAHQIPIPVDRNHMEHADKLVDWLKSYKPEELFDENGTLKPEIAAIIPEGQARMAANPVTNGGKLTKDLITPNIDDYALDNKDHGKEDGSDMTELGKYIRDLIELNKDNKNFRGWGPDETLSNKLGAAFEDTKRQWMEPIHEPNDALLAPQGRIIDSMLSEHMDEGMLEAYNLTGRYGFFASYESFLRVVDSMLTQHFKWLRNSHEETPWRADVPSLNVIASSTAFQQDHNGYSHQDPGIISHLAEKKTEYVRAYLPGDANTLIATFDKAIQSKQLINLIIASKHPRPQWFTMDEAKRLVRDGLGVIDWASTDHGEEPDVVFATAGSEPTTESLAAVSILHARFPEMKIRFINVVDLLKLKKDDPRGLSDAEFDAFFTKDKPVIFAYHAYDDLVKTIFFDRHNHNLHVHGYREEGDITTPFDMRVRNELDRFHLVKAALLATPAYAEKGAHVIQEMNSILDKHHDYIRAEGTDIPEVENWKWTALK

(19) United States
(12) Patent Application Publication
(10) Pub. No.: US 2022/0213512 A1
(43) Pub. Date: Jul. 7, 2022

(54) METHOD FOR CONSTRUCTING THE RECOMBINANT YEASTS FOR PREPARATION OF TYROSOL AND DERIVATIVES AND ITS APPLICATION

(71) Applicant: SHANDONG HENGLU BIOTECH.CO., LTD, Jinan (CN)

(72) Inventors: Xu FANG, Yantai (CN); Wei GUO, Yantai (CN); Shaoli HOU, Yantai (CN)

(21) Appl. No.: 17/264,889

(22) PCT Filed: Mar. 23, 2020
(86) PCT No.: PCT/CN2020/080627

§ 371 Date: Feb. 1, 2021

(30) Foreign Application Priority Data
Apr. 2, 2019 (CN) 201910262724.6
Oct. 29, 2019 (CN) PCT/CN2019/113879
Oct. 7, 2020 (CN) 202010012886.7

(51) Int. Cl. C12P 7/22; C12N 15/81; C12N 15/52; C12N 1/16

(52) U.S. Cl. CPC C12P 7/22; C12N 15/81; C12N 15/52; C12N 1/16; C12N 2510/02; C12Y 401/01025; C12Y 401/02022; C12Y 504/99005; C12N 2800/102; C12Y 103/01012

(57) ABSTRACT

A recombinant yeast is constructed by introducing an expressed gene of exogenous Fructose-6-phosphate phosphoketolase into a modified yeast cell, and the modified yeast cell is a yeast cell with a metabolic pathway for synthesizing tyrosol via Erythrose-4-phosphate and phosphoenolpyruvate. The present invention discloses for the first time that in the process of expressing Fructose-6-phosphate phosphoketolase in a yeast, Fructose-6-phosphate is synthesized into beta-D-Fructose 1,6-bisphosphate and also catalyzed into Erythrose-4-phosphate and Acetyl-phosphate, and Xylulose-5-phosphate is catalyzed into Glyceraldehydes-3-phosphate and Acetyl-phosphate, which change the metabolic flux distribution of carbon in the yeast, enhance the synthesis of Erythrose-4-phosphate as an important intermediate for the biosynthesis of tyrosol, optimize the metabolic pathway for synthesizing tyrosol, and increase the yields of tyrosol and its derivatives such as hydroxytyrosol.

### FIELD OF THE INVENTION

The present invention relates to construction of a recombinant yeast for preparation of tyrosol and derivatives and its application, in particular to a yeast introduced with an expressed gene of exogenous Fructose-6-phosphate phosphoketolase (fxp), and a method for efficiently producing tyrosol and its derivatives by using the strain, belonging to the technical field of microbial genetic engineering.

### BACKGROUND OF THE INVENTION

Tyrosol is a natural antioxidant derived from olive oil, and is a derivative of phenethyl alcohol. The tyrosol as the aglycone substrate of salidroside is a main medicinal active ingredient of Rhodiola plants and a precursor substance of salidroside and hydroxytyrosol. The tyrosol can protect cells from oxidative damage and is a phenolic compound with important industrial value, the tyrosol and its derivatives are synthetic precursors of a variety of organic compounds, and the tyrosol can be used in a pharmaceutical agent. The hydroxytyrosol as a derivative of the tyrosol has a strong antioxidant effect and multiple physiological and medical functions, and the oxidation resistance of the hydroxytyrosol is stronger than that of the tyrosol. The hydroxytyrosol has been widely used in the industries of biomedicine, functional food, etc., and can prevent the occurrence of cardiovascular diseases, osteopenia, etc. At present, the hydroxytyrosol is mainly extracted from olive leaves. Extraction of hydroxytyrosol from plants is expensive and a lot of arable land was occupied.

Synthesis methods using phenethyl alcohol among the chemical methods mostly protect hydroxyl first, and then obtain p-hydroxyphenylethyl alcohol by means of nitration, reduction, diazotization, and hydrolysis, with a yield of 70%. The phenethyl alcohol is expensive and in short supply. Synthesizing the phenethyl alcohol with nitrotoluene is characterized by low cost, but complex steps and low yield. The phenethyl alcohol is synthesized from p-hydroxystyrene, the yield reaches 96%, and the purity is 99%, but the costs of p-hydroxystyrene are relatively high. The preparation of tyrosol by chemical methods is expensive owe to raw material costs and environment-unfriendliness. These problems directly restrict the industrial production of tyrosol with chemical methods. Therefore, biosynthesis of tyrosol and its derivatives has become a research hotspot.

Tyrosol has the following characteristics: chemical name 4-(2-Hydroxyethyl)phenol, molecular formula C₈H₁₀O₂, molecular weight 138.164, CAS number 501-94-0, and structural formula

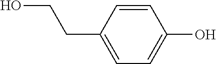

Chinese patent document CN108753636A (application number 201810601213.8) discloses a yeast for producing tyrosol and hydroxytyrosol and a construction method of a recombinant yeast, wherein PcAAS and ADH sequences were introduced into a yeast BY4741 to obtain a PcAAS-ADH recombinant yeast for producing tyrosol; a pdc1 gene knockout cassette and a tyrA expression cassette were introduced into the PcAAS-ADH recombinant yeast to obtain a PcAAS-ADH-Δpdc1-tyrA recombinant yeast for producing tyrosol; and a DNA sequence of HpaBC was introduced into the PcAAS-ADH-Δpdc1-tyrA recombinant yeast to obtain a PcAAS-ADH-HpaBC-Δpdc1-tyrA recombinant yeast for producing hydroxytyrosol. A biosynthesis pathway of tyrosol or hydroxytyrosol was constructed in the BY4741 to increase the output of the tyrosol or hydroxytyrosol. Although this technology can increase the output of tyrosol in the yeast, the output of tyrosol still cannot meet the requirements of industrial production. The synthesis of tyrosol in the yeast is affected by a variety of metabolic pathways, and relevant metabolic pathways have not been fully studied. Therefore, how to prepare the tyrosol with fermentation is still a technical problem at present.

### SUMMARY OF THE INVENTION

Aiming at the deficiencies of the prior method, the present invention provides a novel method for constructing the recombinant yeasts for preparation of tyrosol and derivatives and its application.

The first objective of the present invention is to express, based on the invention patent application (application number 201810601213.8), a gene fragment of Fructose-6-phosphate phosphoketolase, EC 4.1.2.22 (amino acid sequence shown as GenBank: BAF39468.1, SEQ ID No. 1) derived from Bifidobacterium adolescentis No. ATCC 15703 or Fructose-6-phosphate phosphoketolase, EC 4.1.2.22 (amino acid sequence shown as GenBank: KND53308.1, SEQ ID No. 2) derived from Bifidobacterium breve BBRI4 in a yeast, and to construct a new pathway for producing Erythrose-4-phosphate, an important precursor substance for the biosynthesis of tyrosol, by the catalysis of Fructose-6-phosphate, so as to improve the yield of the tyrosol.

The second objective of the present invention is to provide a method for producing hydroxytyrosol.

The third objective of the present invention is to provide a construction method of a yeast for producing tyrosol.

The fourth objective of the present invention is to provide an application of the yeast for producing tyrosol or the construction method in the production of tyrosol.

The fifth objective of the present invention is to provide an application of the yeast for producing tyrosol or the construction method in the production of hydroxytyrosol.

In order to solve the above technical problems, the technical solution of the present invention is as follows:

An application of a recombinant yeast in the production of tyrosol, the recombinant yeast is constructed by introducing an expressed gene of exogenous Fructose-6-phosphate phosphoketolase into a modified yeast cell, and the modified yeast cell is a yeast cell with a metabolic pathway for synthesizing tyrosol via Erythrose-4-phosphate and phosphoenolpyruvate.

According to the present invention, preferably, the modified yeast cell is obtained by integrating an aromatic aldehyde synthase and a fused chorismate mutase T/prephenate dehydrogenase; or the modified yeast cell is obtained by integrating aromatic aldehyde synthases;

According to the present invention, further preferably, the aromatic aldehyde synthase is derived from Petroselinum crispum, the system number of which is EC4.1.1.25; the fused chorismate mutase T is derived from E. coli, the system number of which is EC1.3.1.12; and the prephenate dehydrogenase is derived from E. coli, the system number of which is EC1.3.1.12, EC5.4.99.5.

According to the present invention, preferably, the expressed gene of the Fructose-6-phosphate phosphoketolase is derived from Bifidobacterium adolescentis, Bifidobacterium animalis, Bifidobacterium bifidum, Aspergillus nidulans, Bifidobacterium breve, Bifidobacterium lactis, Clostridium acetobutylicum, Bifidobacterium longum, Bifidobacterium dentium, Leuconostoc mesenteroides, Bifidobacterium mongoliense, Lactobacillus paraplantarum, Lactobacillus plantarum, Bifidobacterium pseudolongum, Candida tropicalis, Cryptococcus neoformans, Cupriavidus necator, Gardnerella vaginalis, Rhodotorula glutinis, Rhodotorula graminis, Saccharomyces pastorianus, etc.

More preferably, the amino acid sequence of the Fructose-6-phosphate phosphoketolase is shown as SEQ ID No. 1 or SEQ ID No. 2, and the nucleotide sequence of the expressed gene is shown as SEQ ID No. 3 or SEQ ID No. 4;

More preferably, the amino acid sequence of the Fructose-6-phosphate phosphoketolase is shown as SEQ ID No. 30, and the nucleotide sequence of the expressed gene is shown as SEQ ID No. 31.

According to the present invention, preferably, the yeast cell is: Saccharomyces cerevisiae, Yarrowia lipolytica, Schizosaccharomyces pombe, Kluyveromyces lactis, Kluyveromyces marxianus, Candida lipolytica, Torulopsis glabrata, Rhodotorula glutinis, Rhodotorula graminis, Saccharomyces pastorianus, Candida tropicalis, Zygosaccharomyces rouxii, Candida glabrata, Torulaspora delbrueckii, Debaryomyces hansenii, Scheffersomyces stipites, Meyerozyma guilliermondii, Lodderomyces elongisporus, Candida albicans, Candida orthopsilosis, Candida metapsilosis, Candida dubliniensis, Clovispora lusitaniae, or Candida auris.

Further preferably, the yeast cell is Saccharomyces cerevisiae, with a strain number CICC1964; and the Kluyveromyces marxianus has a strain number NBRC1777.

According to the present invention, more preferably, the modified yeast cell is obtained by integrating an aromatic aldehyde synthase derived from Petroselinum crispum into a delta12 site of Saccharomyces cerevisiae CICC1964, and substituting a fused chorismate mutase T/prephenate dehydrogenase derived from E. coli for a pdc1 gene of Saccharomyces cerevisiae CICC1964.

A construction method of a recombinant yeast for high tyrosol production includes the following steps:

(1) constructing an expression cassette, which is obtained by fusion of a promoter, a terminator, homologous arms, and an expressed gene of Fructose-6-phosphate phosphoketolase; and

(2) transforming the expression cassette constructed in (1) into a modified yeast cell to obtain the recombinant yeast for high tyrosol production;

wherein the modified yeast cell is a yeast cell with a metabolic pathway for synthesizing tyrosol via Erythrose-4-phosphate and phosphoenolpyruvate.

According to the present invention, more preferably, the modified yeast cell is obtained by integrating an aromatic aldehyde synthase derived from Petroselinum crispum into a delta12 site of Saccharomyces cerevisiae CICC1964, and substituting a fused chorismate mutase T/prephenate dehydrogenase derived from E. coli for a pdc1 gene of Saccharomyces cerevisiae CICC1964.

According to the present invention, more preferably, the modified yeast cell is obtained by integrating an aromatic aldehyde synthase derived from Petroselinum crispum into a delta12 site of Kluyveromyces marxianus NBRC1777, and substituting a fused chorismate mutase T/prephenate dehydrogenase derived from E. coli for a pdc1 gene of Kluyveromyces marxianus NBRC1777.

According to the present invention, preferably, the expressed gene of the Fructose-6-phosphate phosphoketolase in (1) is derived from Bifidobacterium adolescentis, Bifidobacterium animalis, Bifidobacterium bifidum, Aspergillus nidulans, Bifidobacterium breve, Bifidobacterium lactis, Clostridium acetobutylicum, Bifidobacterium longum, Bifidobacterium dentium, Leuconostoc mesenteroides, Bifidobacterium mongoliense, Lactobacillus paraplantarum, Lactobacillus plantarum, Bifidobacterium pseudolongum, Candida tropicalis, Cryptococcus neoformans, Cupriavidus necator, Gardnerella vaginalis, Rhodotorula glutinis, Rhodotorula graminis, Saccharomyces pastorianus, etc.

More preferably, the amino acid sequence of the Fructose-6-phosphate phosphoketolase is shown as SEQ ID No. 1 or SEQ ID No. 2, and the nucleotide sequence of the expressed gene is shown as SEQ ID No. 3 or SEQ ID No. 4.

More preferably, the amino acid sequence of the Fructose-6-phosphate phosphoketolase is shown as SEQ ID No. 30, and the nucleotide sequence of the expressed gene is shown as SEQ ID No. 31.

According to the present invention, preferably, the homologous arms in (1) are forward and reverse 500 bp gene fragments of a prephenate dehydrogenase gene pha2 amplified with primers using a genome of Saccharomyces cerevisiae CICC1964 or Kluyveromyces marxianus NBRC1777 as a template, wherein the nucleotide sequences of the amplification primers for the forward homologous arm are respectively shown as SEQ ID No. 5 and SEQ ID No. 6; and the nucleotide sequences of the amplification primers for the reverse homologous arm are respectively shown as SEQ ID No. 7 and SEQ ID No. 8;

According to the present invention, preferably, the promoter in (1) is a promoter tpi1 amplified with primers using a genome of Saccharomyces cerevisiae CICC1964 or Kluyveromyces marxianus NBRC1777 as a template, and the nucleotide sequences of the amplification primers for the promoter tpi1 are respectively shown as SEQ ID No. 5 and SEQ ID No. 6;

According to the present invention, preferably, the terminator in (1) is a terminator gpm1 amplified with primers using a genome of Saccharomyces cerevisiae CICC1964 or Kluyveromyces marxianus NBRC1777 as a template, and the nucleotide sequences of the amplification primers for the terminator gpm1 are respectively shown as SEQ ID No. 9 and SEQ ID No. 10;

According to the present invention, preferably, the modified yeast cell in (2) is obtained by integrating an aromatic aldehyde synthase and a fused chorismate mutase T/prephenate dehydrogenase; or the modified yeast cell is obtained by integrating aromatic aldehyde synthases;

According to the present invention, further preferably, in (2), the aromatic aldehyde synthase is derived from Petroselinum crispum, the system number of which is EC4.1.1.25; and the fused chorismate mutase T/prephenate dehydrogenase is derived from E. coli, the system number of which is EC1.3.1.12, EC 5.4.99.5.

According to the present invention, preferably, the yeast cell in (2) is: Saccharomyces cerevisiae, Yarrowia lipolytica, Schizosaccharomyces pombe, Kluyveromyces lactis, Kluyveromyces marxianus, Candida lipolytica, Torulopsis glabrata, Rhodotorula glutinis, Rhodotorula graminis, Saccharomyces pastorianus, Candida tropicalis, Zygosaccharomyces rouxii, Candida glabrata, Torulaspora delbrueckii, Debaryomyces hansenii, Scheffersomyces stipites, Meyerozyma guilliermondii, Lodderomyces elongisporus, Candida albicans, Candida orthopsilosis, Candida metapsilosis, Candida dubliniensis, Clovispora lusitaniae, Candida auris, etc.

Further preferably, the yeast cell is Saccharomyces cerevisiae, with a strain number CICC1964; and the Kluyveromyces marxianus has a strain number NBRC1777.

Further preferably, the modified yeast cell in (2) is obtained by integrating an aromatic aldehyde synthase derived from Petroselinum crispum into a delta12 site of Saccharomyces cerevisiae CICC1964.

According to the present invention, more preferably, the modified yeast cell in (2) is obtained by integrating an aromatic aldehyde synthase derived from Petroselinum crispum into a delta12 site of Saccharomyces cerevisiae CICC1964, and substituting a fused chorismate mutase T/prephenate dehydrogenase derived from E. coli for a pdc1 gene of Saccharomyces cerevisiae CICC1964.

Further preferably, the modified yeast cell in (2) is obtained by integrating an aromatic aldehyde synthase derived from Petroselinum crispum into a delta12 site of Kluyveromyces marxianus NBRC1777.

According to the present invention, more preferably, the modified yeast cell in (2) is obtained by integrating an aromatic aldehyde synthase derived from Petroselinum crispum into a delta12 site of Kluyveromyces marxianus NBRC1777, and substituting a fused chorismate mutase T/prephenate dehydrogenase derived from E. coli for a pdc1 gene of Kluyveromyces marxianus NBRC1777.

A recombinant yeast for high tyrosol production constructed by the above method.

An application of the above-mentioned recombinant yeast for high tyrosol production in fermentation preparation of tyrosol.

According to the present invention, preferably, the fermentation medium for fermentation contains at least one or a combination of two or more of glucose, fructose and sucrose, and tyrosine.

An application of the above-mentioned recombinant yeast for high tyrosol production in fermentation preparation of hydroxytyrosol.

According to the present invention, preferably, after the above-mentioned recombinant yeast for high tyrosol production is fermented to prepare tyrosol, hydroxytyrosol is obtained through a hydroxylase reaction.

According to the present invention, preferably, the tyrosol obtained by fermenting the above-mentioned recombinant yeast for high tyrosol production is catalyzed by E. coli of overexpressed 4-hydroxyphenylacetate hydroxylase to obtain the hydroxytyrosol.

According to the present invention, further preferably, the fermentation medium for fermentation contains at least one or a combination of two or more of glucose, fructose and sucrose, and tyrosine.

Beneficial Effects of the Present Invention:

1. The present invention discloses for the first time that in the expression process of a yeast, Fructose-6-phosphate is synthesized into beta-D-Fructose 1,6-bisphosphate and also catalyzed into Erythrose-4-phosphate and Acetyl-phosphate, and Xylulose-5-phosphate is catalyzed into Glyceraldehydes-3-phosphate and Acetyl-phosphate, which change the metabolic flux distribution of carbon in the yeast, enhance the synthesis of Erythrose-4-phosphate as an important intermediate for the biosynthesis of tyrosol, optimize the metabolic pathway for synthesizing tyrosol, and increase the yields of tyrosol and its derivatives such as hydroxytyrosol;

2. The expressed gene of exogenous Fructose-6-phosphate phosphoketolase is introduced into the modified yeast cell to obtain a recombinant yeast, which can increase the yield of tyrosol; and the tyrosol is catalyzed by E. coli of overexpressed 4-hydroxyphenylacetate hydroxylase to obtain hydroxytyrosol.

3. The present invention provides a novel and environment-friendly biological preparation technology for tyrosol and hydroxytyrosol, which lays a foundation for the large-scale industrial production of tyrosol and hydroxytyrosol, and has important economic value and social benefits.

### BRIEF DESCRIPTION OF THE DRAWING

FIG. 1 is a structural diagram of a recombinant plasmid pUG6 in Embodiment 1.

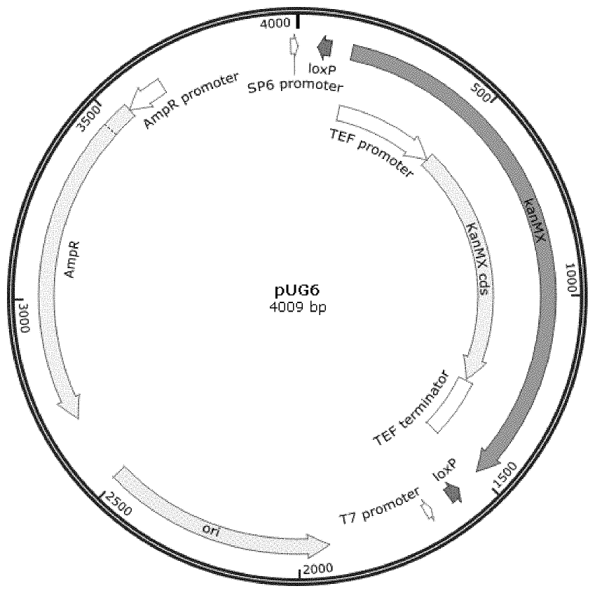

### DETAILED DESCRIPTION OF EMBODIMENTS

It should be noted that the following detailed descriptions are exemplary and are intended to provide further descriptions of the present application. All technical and scientific terms used herein have the same meanings as commonly understood by those ordinary skilled in the art to which the present application belongs, unless specified otherwise.

It should be noted that terms used herein are intended to describe specific implementation modes only rather than to limit the exemplary implementation modes according to the present application. As used herein, the singular form is also intended to comprise the plural form unless otherwise indicated in the context. In addition, it should be understood that when the terms “contain” and/or “comprise” are used in the description, they are intended to indicate the presence of features, steps, operations, devices, components and/or combinations thereof.

The present invention will be further illustrated below in conjunction with embodiments:

In the following embodiments, E. coli BL21 and expression vector pET-28a are commercially available. The experimental methods unmarked with specific conditions in the following embodiments were carried out according to conventional conditions, for example, the conditions described in Molecular Cloning: A Laboratory Manual, or according to the conditions recommended by the manufacturers of corresponding biological reagents. The PCR amplification reaction process may be a conventional PCR amplification reaction process.

Saccharomyces cerevisiae CICC1964, purchased from China Center of Industrial Culture Collection, with a strain number CICC1964, is a known non-collected strain;

According to the method of the invention patent application (application number 201810601213.8), the aromatic aldehyde synthase (AAS, EC4.1.1.25) derived from Petroselinum crispum was integrated into a delta12 site of Saccharomyces cerevisiae CICC1964, and a fused chorismate mutase T/prephenate dehydrogenase (TyrA, EC1.3.1.12, EC 5.4.99.5) derived from E. coli was substituted for a pdc1 gene of the Saccharomyces cerevisiae CICC1964 to obtain an SC-1 strain.

### Embodiment 1

Construction of Bafxpk and Bbfxpk Expression Cassettes

Codon optimization was performed on amino acid sequences of SEQ ID No. 1 and SEQ ID No. 2 according to the codon preference of a host Saccharomyces cerevisiae to obtain optimized nucleotide sequences SEQ ID No. 3 and SEQ ID No. 4 corresponding to SEQ ID No. 1 and SEQ ID No. 2 for gene synthesis. An expressed gene of a target gene Fructose-6-phosphate phosphoketolase was obtained by amplification with primer pairs Bafxpk-F/Bafxpk-R and Bbfxpk-F/Bbfxpk-R via a Phanta Max High-Fidelity DNA polymerase from Vazyme (the nucleotide sequence of the Bafxpk fragment was shown as SEQ ID No. 3, and the nucleotide sequence of the Bbfxpk fragment was shown as SEQ ID No. 4). By using a genome of Saccharomyces cerevisiae CICC1964 as a template, DNA fragments of forward and reverse homologous arms were amplified with primer pairs U-F/U-R and D-F/D-R via PCR, and fragments of a promoter tpi1 and a terminator gpm1 were amplified with primer pairs Ptpi1-F/Ptpi1-R and Tgpm1-F/Tgpm1-R. A DNA fragment of a resistance gene KanMX4 was amplified with primer pairs G418-F/G418-R via PCR, using DNA with a recombinant plasmid pUG6 (as shown in FIG.) of a geneticin resistance gene KanMX4 (the nucleotide sequence was shown as SEQ ID No. 24) as a template. After the size of a band was verified by agarose gel electrophoresis to be correct, the band was cut, and gene fragments were recovered with an OMEGA gel extraction kit. The primers for PCR amplification were as follows:

The sequences of the primer pair Bafxpk-F/Bafxpk-R were SEQ ID No. 13 and SEQ ID No. 14; the sequences of the primer pair Bbfxpk-F/Bbfxpk-R were SEQ ID No. 15 and SEQ ID No. 16; the sequences of the primer pair U-F/U-R were SEQ ID No. 5 and SEQ ID No. 6; the sequences of the primer pair D-F/D-R were SEQ ID No. 7 and SEQ ID No. 8; the sequences of the primer pair Ptpi1-F/Ptpi1-R were SEQ ID No. 9 and SEQ ID No. 10; the sequences of the primer pair Tgpm1-F/Tgpm1-R were SEQ ID No. 11 and SEQ ID No. 12; and the sequences of the primer pair G418-F/G418-R were SEQ ID No. 17 and SEQ ID No. 18;

The target gene fragments were amplified with Phanta Max High-Fidelity DNA polymerase, each fragment was ensured to have a 50 bp homologous sequence with an adjacent fragment, a PCR product was recovered with a DNA fragment gel recovery kit after the gel electrophoresis, and DNA concentration was measured. Then, the purified DNA fragments with homologous sequences were fused by a fusion PCR method:

(1) The fragments were ligated by using Phanta Max High-Fidelity DNA polymerase. The reaction system was shown as Table 1:

TABLE 1
Reaction system

| Composition | Adding amount (μL) |
|---|---|
| DNA fragments | 200 ng each |
| 2 x Phanta Max Buffer 2 | 2 |
| dNTP Mix (10 Mm each) | 0.2 |
| Phanta Max High-Fidelity | 0.5 |
| DNA polymerase | |
| dd H₂O | Make up to 20 μL |
| Total | 25 |

The above reagents were added to a PCR tube, and the reaction conditions were shown as Table 2:

TABLE 2
Reaction conditions

| | |
|---|---|
| Reaction temperature | Reaction Time |

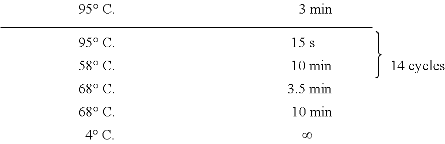

(2) By using the PCR product of (1) as a PCR amplification template, the obtained target fragments were amplified with a primer pair Yzaw-F/Yzaw-R via Phanta Max High-Fidelity DNA from Vazyme. After the size of the band was verified by agarose gel electrophoresis to be correct, the band was cut, and DNA fragments, i.e., DNA fragments of Bafxpk and Bbfxpk expression cassettes, were recovered with the OMEGA gel extraction kit. The primers for PCR amplification were as follows:

The sequence of Yzaw-F was SEQ ID No. 19; the sequence of Yzaw-R was SEQ ID No. 20;

(3) The DNA fragments of the Bafxpk and Bbfxpk expression cassettes obtained in (2) were verified by sequencing.

### Embodiment 2

Construction of Bafxpk and Bbfxpk Heterologous Expression Strains, Taking Saccharomyces cerevisiae as an Example:

Saccharomyces cerevisiae tyrosol synthesis strain CICC1964 was transformed by a PEG/LiAc method, resistance G418 was added to a medium for screening the strain, a genome was extracted, PCR verification was performed by using a primer pair Yzaw-F/Yzaw-R, and SC-bafxpk and SC-bbfxpk strains were obtained.

### Embodiment 3

Fermentation of Microorganisms for Synthesizing Tyrosol, Taking Saccharomyces cerevisiae as an Example:

The yeasts were picked from a plate of strains CICC1964, SC-bafxpk and SC-bbfxpk for producing tyrosol, inoculated to a 5 mL YPD liquid medium, cultured under the conditions of 30-32° C. and 200 rpm for 24 h, and transferred to a 50 mL YPD liquid medium, wherein the initial inoculation OD₆₀₀ was 0.2; the broths were cultured under the conditions of 30° C. and 200 rpm for 12 h, and then transferred to a 100 mL YPD liquid medium, wherein the initial inoculation OD₆₀₀ was 0.2, and the medium contained a carbon source such as 2% glucose or 2% sucrose or 2% glucose and 1% tyrosine; and after 24 hours of culture, the carbon source such as 2% glucose or 2% sucrose or 2% glucose and 1% tyrosine was added again for a total of 72 hours of fermentation. The concentration of tyrosol in the fermentation broth was tested by the HPLC method reported in the literature (Satoh et al., Journal of Agricultural and Food Chemistry, 60, 979-984, 2012). The yields of tyrosol under different carbon source culture conditions were shown in Table 3.

TABLE 3
Yields of tyrosol after 72 hours of fermentation
under different carbon sources

| | Glucose | Sucrose | Glucose + tyrosine |
|---|---|---|---|
| Strain (mg/L) | (mg/L) | (mg/L) | (mg/L) |
| SC-bafxp | 3103.16 | 3240.37 | 3526.18 |
| SC-bbfxpk | 3218.41 | 3400.23 | 3684.27 |
| SC-1 | 890.38 | 906.14 | 113.75 |

### Embodiment 4

Method for Obtaining Gene HpaBC and E. coli Expression Vector

A DNA sequence gene cluster of 4-hydroxyphenylacetate 3-hydroxylase (HpaBC, enzyme system number EC 1.5.1.37) derived from E. coli was overexpressed in E. coli, and the tyrosol was catalyzed into hydroxytyrosol by using whole E. coli cells.

An amino acid sequence gene cluster of the 4-hydroxyphenylacetate 3-hydroxylase included: the amino acid sequence of 4-hydroxyphenylacetate hydroxylase (HpaB) was SEQ ID No. 21, and the corresponding nucleotide sequence was SEQ ID NO. 23; the amino acid sequence of 4-hydroxyphenylacetate hydroxylase (HpaC) was SEQ ID No. 22, and the corresponding nucleotide sequence was SEQ ID NO. 24.

An E. coli DE3 genome was extracted as a template by using a bacterial genome kit, the SEQ ID No. 23 and the SEQ ID No. 24 were amplified with primer pairs hpaB-F/hpaB-R and hpaC-F/hpaC-R, respectively, and sequencing verification was performed. The sequence of hpaB-F was SEQ ID No. 25; the sequence of hpaB-R was SEQ ID No. 26; the sequence of hpaC-F was SEQ ID No. 27; and the sequence of hpaC-R was SEQ ID No. 28.

E. coli containing a pET-28a empty vector was cultured with pET-28a as an expression vector, pET-28a plasmids were extracted by using a bacterial plasmid extraction kit, and an expression vector pEThpaBC (SEQ ID NO. 29) was constructed by using a conventional molecular biology method. Then, the pEThpaBC was transformed into an E. coli expression vector BL21, and kanamycin was used as a selection marker to obtain monoclonal BL21-pEThpaBC.

The BL21-pEThpaBC was cultured by shaking, and the expression of HPAB/C was induced by using 1 mM IPTG.

The obtained BL21-pEThpaBC strain was added to the medium of Embodiment 3. After reacting for 3 hours, the yields of hydroxytyrosol obtained were tested. The results were shown in Table 4:

TABLE 4
Yields of hydroxytyrosol after adding BL21-pEThpaBC strain
to the medium shown in Table 4 and mixing for 3 hours

| | Glucose | Sucrose | Glucose + tyrosine |
|---|---|---|---|
| Strain (mg/L) | (mg/L) | (mg/L) | (mg/L) |
| SC-bafxp | 3010.34 | 2989.15 | 3268.39 |
| SC-bbfxpk | 3156.81 | 3008.51 | 3409.83 |
| SC-1 | 840.28 | 850.31 | 101.25 |

### Embodiment 5

Kluyveromyces marxianus with a strain number NBRC1777 was used. According to the method of the invention patent application (application number 201810601213.8), an aromatic aldehyde synthase (AAS, EC4.1.1.25) derived from Petroselinum crispum was integrated into a delta12 site of the Kluyveromyces marxianus, and a fused chorismate mutase T/prephenate dehydrogenase (TyrA, EC1.3.1.12, EC 5.4.99.5) derived from E. coli was substituted for a pdc1 gene of the Kluyveromyces marxianus to obtain a Kluyveromyces marxianus strain with a metabolic pathway for synthesizing tyrosol via Erythrose-4-phosphate and phosphoenolpyruvate.

KM-bafxp and KM-bbfxp strains were prepared via Fructose-6-phosphate phosphoketolase (EC 4.1.2.22) according to the methods described in Embodiments 1-2 of the present invention, and the difference was that the culture temperature of Kluyveromyces marxianus cells was 42-49° C.

### Embodiment 6

Fructose-6-phosphate phosphoketolase (bdfxp, GenBank: BAQ26957.1) derived from Bifidobacterium dentium was used, with an amino acid sequence shown as SEQ ID No. 30. Tyrosol and hydroxytyrosol were prepared according to the methods described in Embodiment 3 and Embodiment 4. The sequences of a primer pair Bdfxp-F/Bdfxp-R used were SEQ ID No. 32 and SEQ ID No. 33. The results were similar to those of Embodiment 3 and Embodiment 4, i.e., the yields of tyrosol and hydroxytyrosol were also increased. The specific results were as follows:

TABLE 5
Yields of tyrosol after 72 hours of fermentation
under different carbon sources

| | Glucose | Sucrose | Glucose + tyrosine |
|---|---|---|---|
| Strain (mg/L) | (mg/L) | (mg/L) | (mg/L) |
| SC-bdfxp | 3194.84 | 3215.46 | 3570.07 |

TABLE 6
Yields of hydroxytyrosol after adding BL21-pEThpaBC strain
to the medium shown in Table 6 and mixing for 3 hours

| | Glucose | Sucrose | Glucose + tyrosine |
|---|---|---|---|
| Strain (mg/L) | (mg/L) | (mg/L) | (mg/L) |
| SC-bdfxp | 3209.89 | 3146.29 | 3310.81 |

### Embodiment 7

According to the methods described in Embodiments 1-2, an aromatic aldehyde synthase (AAS, EC4.1.1.25) derived from Petroselinum crispum was integrated into a delta12 site of Saccharomyces cerevisiae CICC1964, and Fructose-6-phosphate phosphoketolase (EC 4.1.2.22) as shown in SEQ ID NO. 4 was introduced into the yeast to obtain an SC-bbfxpk-AAS strain. The result was similar to the tyrosol yield of the SC-bbfxpk strain in Embodiment 3.

The obtained BL21-pEThpaBC strain was added to the prepared medium containing tyrosol. After reacting for 3 hours, the yield of hydroxytyrosol obtained was tested, and the result was also similar to that of the SC-bbfxpk strain described in Embodiment 4. It showed that similar yields of tyrosol and hydroxytyrosol can also be obtained without introducing a fused chorismate mutase T/prephenate dehydrogenase (TyrA, EC1.3.1.12, EC 5.4.99.5) gene into the yeast. The specific results were shown in Table 7 and Table 8:

TABLE 7
Yields of tyrosol after 72 hours of fermentation
under different carbon sources

| | Glucose | Sucrose | Glucose + tyrosine |
|---|---|---|---|
| Strain (mg/L) | (mg/L) | (mg/L) | (mg/L) |
| SC-bbfxpk- | 3291.76 | 3402.13 | 3702.09 |
| AAS | | | |
| SC-bbfxpk | 3218.41 | 3400.23 | 3684.27 |

TABLE 8
Yields of hydroxytyrosol after adding BL21-pEThpaBC strain
to the medium shown in Table 8 and mixing for 3 hours

| | Glucose | Sucrose | Glucose + tyrosine |
|---|---|---|---|
| Strain (mg/L) | (mg/L) | (mg/L) | (mg/L) |
| SC-bbfxpk- | 3202.65 | 3015.77 | 3432.72 |
| AAS | | | |
| SC-bbfxpk | 3156.81 | 3008.51 | 3409.83 |

### Embodiment 8

According to the method described in Embodiment 6, gene fragments corresponding to Fructose-6-phosphate phosphoketolase (baifxp, GenBank: WP_052826255.1) with an amino acid sequence SEQ ID No. 100 derived from Bifidobacterium animalis, Fructose-6-phosphate phosphoketolase (bbifxp, GenBank: WP_047289945.1) with an amino acid sequence SEQ ID No. 101 derived from Bifidobacterium bifidum, Fructose-6-phosphate phosphoketolase (blafxp, GenBank: CAC29121.1) with an amino acid sequence SEQ ID No. 102 derived from Bifidobacterium lactis, Fructose-6-phosphate phosphoketolase (blofxp, GenBank: PWH09343.1) with an amino acid sequence SEQ ID No. 103 derived from Bifidobacterium longum, Fructose-6-phosphate phosphoketolase (bmfxp, GenBank: CAC29121.1) with an amino acid sequence SEQ ID No. 104 derived from Bifidobacterium mongoliense, Fructose-6-phosphate phosphoketolase (bpfxp, GenBank: WP_034883174.1) with an amino acid sequence SEQ ID No. 105 derived from Bifidobacterium pseudolongum, Fructose-6-phosphate phosphoketolase (Anfxp, GenBank: CBF76492.1) with an amino acid sequence SEQ ID No. 106 derived from Aspergillus nidulans, Fructose-6-phosphate phosphoketolase (Cafxp, GenBank: KHD36088.1) with an amino acid sequence SEQ ID No. 107 derived from Clostridium acetobutylicum, Fructose-6-phosphate phosphoketolase (Lmfxp, GenBank: AAV66077.1) with an amino acid sequence SEQ ID No. 108 derived from Leuconostoc mesenteroides, Fructose-6-phosphate phosphoketolase (Lprfxp, GenBank: AL004878.1) with an amino acid sequence SEQ ID No. 109 derived from Lactobacillus paraplantarum, and Fructose-6-phosphate phosphoketolase (Lplfxp, GenBank: KRU19755.1) with an amino acid sequence SEQ ID No. 110 derived from Lactobacillus plantarum, were transformed into modified Saccharomyces cerevisiae; modified strains SC-baifxp, SC-bbifxp, SC-blafxp, SC-blofxp, SC-bmfxp, SC-bpfxp, SC-Anfxp, SC-Cafxp, SC-Lmfxp, SC-Lprfxp, and SC-Lplfxp were obtained, respectively; and the modified Saccharomyces cerevisiae was obtained by integrating an aromatic aldehyde synthase derived from Petroselinum crispum into a delta12 of Saccharomyces cerevisiae CICC1964. The results were similar to those of Embodiment 3 and Embodiment 4, i.e., the yields of tyrosol and hydroxytyrosol were also increased. The specific results were shown in Table 9 and Table 10.

TABLE 9
Yields of tyrosol after 72 hours of fermentation
under different carbon sources

| | Glucose | Sucrose | Glucose + tyrosine |
|---|---|---|---|
| Strain (mg/L) | (mg/L) | (mg/L) | (mg/L) |
| SC-baifxp | 1484.214 | 1425.86 | 1625.11 |
| SC-bbifxp | 1522.71 | 1563.47 | 1699.25 |
| SC-blafxp | 1658.23 | 1681.93 | 1821.61 |
| SC-blofxp | 1828.46 | 1900.84 | 2134.61 |
| SC-bmfxp | 1400.19 | 1456.91 | 1611.88 |
| SC-bpfxp | 1233.41 | 1290.41 | 1390.42 |
| SC-Anfxp | 927.04 | 940.71 | 1004.84 |
| SC-Cafxp | 1604.22 | 1612.88 | 1824.61 |
| SC-Lmfxp | 956.17 | 943.45 | 945.21 |
| SC-Lprfxp | 1200.02 | 1210.32 | 1423.56 |
| SC-Lplfxp | 1128.23 | 1194.16 | 1256.23 |

TABLE 10
Yields of hydroxytyrosol after adding BL21-pEThpaBC strain
to the medium shown in Table 10 and mixing for 3 hours

| | Glucose | Sucrose | Glucose + tyrosine |
|---|---|---|---|
| Strain (mg/L) | (mg/L) | (mg/L) | (mg/L) |
| SC-baifxp | 1249.89 | 1246.78 | 1319.71 |
| SC-bbifxp | 1386.09 | 1364.21 | 1402.34 |
| SC-blafxp | 1423.02 | 1401.32 | 1634.09 |
| SC-blofxp | 1614.02 | 1715.56 | 1973.21 |
| SC-bmfxp | 1267.01 | 1301.11 | 1472.01 |
| SC-bpfxp | 1011.33 | 1199.21 | 1187.43 |
| SC-Anfxp | 871.21 | 877.09 | 903.89 |
| SC-Cafxp | 1500.09 | 1490.36 | 1614.70 |
| SC-Lmfxp | 799.23 | 789.02 | 801.90 |
| SC-Lprfxp | 1000.82 | 1060.11 | 1225.43 |
| SC-Lplfxp | 997.13 | 909.61 | 1023.31 |

From the above results, when the above exogenous Fructose-6-phosphate phosphoketolases were expressed in yeast cells, the yields were different and lower than those using the Fructose-6-phosphate phosphoketolases derived from Bifidobacterium adolescentis, Bifidobacterium breve and Bifidobacterium dentium in Embodiment 3, Embodiment 4 and Embodiment 5, but compared with original strain, the yields of tyrosol and hydroxytyrosol were significantly increased.

Based on the results of Embodiment 7, the inventors simultaneously transformed gene fragments corresponding to the above Fructose-6-phosphate phosphoketolases into SC-1 strain obtained according to the method of the invention patent application (application number 201810601213.8), i.e.: an aromatic aldehyde synthase (AAS, EC4.1.1.25) derived from Petroselinum crispum was integrated into a delta12 site of Saccharomyces cerevisiae CICC1964, and a fused chorismate mutase T/prephenate dehydrogenase (TyrA, EC1.3.1.12, EC 5.4.99.5) derived from E. coli is substituted for a pdc1 gene of the Saccharomyces cerevisiae CICC1964; the results showed that the results of baifxp, bbifxp, blafxp, blofxp, bmfxp, bpfxp, Anfxp, Cafxp, Lmfxp, Lprfxp, and Lplfxp were similar to the results of bbfxpk in Embodiment 7, indicating whether the fused chorismate mutase T/prephenate dehydrogenase (TyrA, EC1.3.1.12, EC 5.4.99.5) gene was introduced into the yeast, the yields of tyrosol and hydroxytyrosol were affected a little, with universality.

Result Analysis

Based on the above results, it can be found by those skilled in the art that when exogenous Fructose-6-phosphate phosphoketolase was expressed in yeast cells, the yield of tyrosol can be significantly increased, and this phenomenon was not limited to one source of Fructose-6-phosphate phosphoketolase and specific yeast cells; it can be expected by those skilled in the art through the technical teaching of the present invention that, after the Fructose-6-phosphate phosphoketolase was expressed in the yeast cells with metabolic pathways for synthesizing tyrosol via Erythrose-4-phosphate and phosphoenolpyruvate, because the expression of the metabolic exogenous Fructose-6-phosphate phosphoketolase affected the sugar metabolism pathways of the yeast cells, the production of tyrosol products was promoted, and the technical effects described in the present application can be achieved.

Described above are merely preferred embodiments of the present application, and the present application is not limited thereto. Various modifications and variations may be made to the present application for those skilled in the art. Any modifications, equivalent substitutions, improvements and the like made within the spirit and principle of the present application shall fall within the protection scope of the present application.

1. A method for producing tyrosol with a recombinant yeast, wherein the recombinant yeast is constructed by introducing an expressed gene of exogenous Fructose-6-phosphate phosphoketolase into a modified yeast cell, and the modified yeast cell is a yeast cell with a metabolic pathway for synthesizing tyrosol via Erythrose-4-phosphate and phosphoenolpyruvate.

2. The method according to claim 1, wherein the modified yeast cell is obtained by integrating an aromatic aldehyde synthase and a fused chorismate mutase T/prephenate dehydrogenase; or the modified yeast cell is obtained by integrating aromatic aldehyde synthases;
further preferably, the aromatic aldehyde synthase is derived from Petroselinum crispum, the system number of which is EC4.1.1.25; the fused chorismate mutase T is derived from E. coli, the system number of which is EC1.3.1.12 and EC5.4.99.5; and the prephenate dehydrogenase is derived from E. coli, the system number of which is EC5.4.99.5.

3. The method according to claim 1, wherein the expressed gene of the Fructose-6-phosphate phosphoketolase is derived from Bifidobacterium adolescentis, Bifidobacterium animalis, Bifidobacterium bifidum, Aspergillus nidulans, Bifidobacterium breve, Bifidobacterium lactis, Clostridium acetobutylicum, Bifidobacterium longum, Bifidobacterium dentium, Leuconostoc mesenteroides, Bifidobacterium mongoliense, Lactobacillus paraplantarum, Lactobacillus plantarum, Bifidobacterium pseudolongum, Candida tropicalis, Cryptococcus neoformans, Cupriavidus necator, Gardnerella vaginalis, Rhodotorula glutinis, Rhodotorula graminis, Saccharomyces pastorianus, etc.;
more preferably, the amino acid sequence of the Fructose-6-phosphate phosphoketolase is shown as SEQ ID No. 1 or SEQ ID No. 2, and the nucleotide sequence of the expressed gene is shown as SEQ ID No. 3 or SEQ ID No. 4;
more preferably, the amino acid sequence of the Fructose-6-phosphate phosphoketolase is shown as SEQ ID No. 30, and the nucleotide sequence of the expressed gene is shown as SEQ ID No. 31.

4. The method according to claim 1, wherein the yeast cell is: Saccharomyces cerevisiae, Yarrowia lipolytica, Schizosaccharomyces pombe, Kluyveromyces lactis, Kluyveromyces marxianus, Candida lipolytica, Torulopsis glabrata, Rhodotorula glutinis, Rhodotorula graminis, Saccharomyces pastorianus, Candida tropicalis, Zygosaccharomyces rouxii, Candida glabrata, Torulaspora delbrueckii, Debaryomyces hansenii, Scheffersomyces stipites, Meyerozyma guilliermondii, Lodderomyces elongisporus, Candida albicans, Candida orthopsilosis, Candida metapsilosis, Candida dubliniensis, Clovispora lusitaniae, Candida auris, etc.;
further preferably, the yeast cell is Saccharomyces cerevisiae, with a strain number CICC1964; and the Kluyveromyces marxianus has a strain number NBRC1777;
further preferably, the modified yeast cell is obtained by integrating an aromatic aldehyde synthase derived from Petroselinum crispum into a delta12 site of Saccharomyces cerevisiae CICC1964;
more preferably, the modified yeast cell is obtained by integrating an aromatic aldehyde synthase derived from Petroselinum crispum into a delta12 site of Saccharomyces cerevisiae CICC1964, and substituting a fused chorismate mutase T/prephenate dehydrogenase derived from E. coli for a pdc1 gene of Saccharomyces cerevisiae CICC1964;
further preferably, the modified yeast cell is obtained by integrating an aromatic aldehyde synthase derived from Petroselinum crispum into a delta12 site of Kluyveromyces marxianus NBRC1777;
more preferably, the modified yeast cell is obtained by integrating an aromatic aldehyde synthase derived from Petroselinum crispum into a delta12 site of Kluyveromyces marxianus NBRC1777, and substituting a fused chorismate mutase T/prephenate dehydrogenase derived from E. coli for a pdc1 gene of Kluyveromyces marxianus NBRC1777.

5. A process for constructing a recombinant yeast for high tyrosol production, comprising the following steps:
(1) constructing an expression cassette, which is obtained by fusion of a promoter, a terminator, homologous arms, and an expressed gene of Fructose-6-phosphate phosphoketolase; and
(2) transforming the expression cassette constructed in (1) into a modified yeast cell to obtain the recombinant yeast for high tyrosol production;
wherein the modified yeast cell is a yeast cell with a metabolic pathway for synthesizing tyrosol via Erythrose-4-phosphate and phosphoenolpyruvate.

6. The process according to claim 5, wherein the expressed gene of the Fructose-6-phosphate phosphoketolase in (1) is derived from Bifidobacterium adolescentis, Bifidobacterium animalis, Bifidobacterium bifidum, Aspergillus nidulans, Bifidobacterium breve, Bifidobacterium lactis, Clostridium acetobutylicum, Bifidobacterium longum, Bifidobacterium dentium, Leuconostoc mesenteroides, Bifidobacterium mongoliense, Lactobacillus paraplantarum, Lactobacillus plantarum, Bifidobacterium pseudolongum, Candida tropicalis, Cryptococcus neoformans, Cupriavidus necator, Gardnerella vaginalis, Rhodotorula glutinis, Rhodotorula graminis, Saccharomyces pastorianus, etc.;
more preferably, the amino acid sequence of the Fructose-6-phosphate phosphoketolase is shown as SEQ ID No. 1 or SEQ ID No. 2, and the nucleotide sequence of the expressed gene is shown as SEQ ID No. 3 or SEQ ID No. 4;
more preferably, the amino acid sequence of the Fructose-6-phosphate phosphoketolase is shown as SEQ ID No. 30, and the nucleotide sequence of the expressed gene is shown as SEQ ID No. 31;
preferably, the homologous arms in (1) are forward and reverse 500 bp gene fragments of a prephenate dehydrogenase gene pha2 amplified with primers using a genome of Saccharomyces cerevisiae CICC1964 or Kluyveromyces marxianus NBRC1777 as a template, wherein the nucleotide sequences of the amplification primers for the forward homologous arm are respectively shown as SEQ ID No. 5 and SEQ ID No. 6; and the nucleotide sequences of the amplification primers for the reverse homologous arm are respectively shown as SEQ ID No. 7 and SEQ ID No. 8;
preferably, the promoter in (1) is a promoter tpi1 amplified with primers using a genome of Saccharomyces cerevisiae CICC1964 or Kluyveromyces marxianus NBRC1777 as a template, and the nucleotide sequences of the amplification primers for the promoter tpi1 are respectively shown as SEQ ID No. 9 and SEQ ID No. 10;
preferably, the terminator in (1) is a terminator gpm1 amplified with primers using a genome of Saccharomyces cerevisiae CICC1964 or Kluyveromyces marxianus NBRC1777 as a template, and the nucleotide sequences of the amplification primers for the terminator gpm1 are respectively shown as SEQ ID No. 11 and SEQ ID No. 12;
preferably, the modified yeast cell in (2) is obtained by integrating an aromatic aldehyde synthase and a fused chorismate mutase T/prephenate dehydrogenase; or the modified yeast cell is obtained by integrating aromatic aldehyde synthases;
further preferably, in (2), the aromatic aldehyde synthase is derived from Petroselinum crispum, the system number of which is EC4.1.1.25; the fused chorismate mutase T/prephenate dehydrogenase is derived from E. coli, the system number of which is EC1.3.1.12, EC 5.4.99.5;
preferably, the yeast cell in (2) is: Saccharomyces cerevisiae, Yarrowia lipolytica, Schizosaccharomyces pombe, Kluyveromyces lactis, Kluyveromyces marxianus, Candida lipolytica, Torulopsis glabrata, Rhodotorula glutinis, Rhodotorula graminis, Saccharomyces pastorianus, Candida tropicalis, Zygosaccharomyces rouxii, Candida glabrata, Torulaspora delbrueckii, Debaryomyces hansenii, Scheffersomyces stipites, Meyerozyma guilliermondii, Lodderomyces elongisporus, Candida albicans, Candida orthopsilosis, Candida metapsilosis, Candida dubliniensis, Clavispora lusitaniae, Candida auris, etc.;
further preferably, the yeast cell is Saccharomyces cerevisiae, with a strain number CICC1964; and the Kluyveromyces marxianus has a strain number NBRC1777;
further preferably, the modified yeast cell in (2) is obtained by integrating an aromatic aldehyde synthase derived from Petroselinum crispum into a delta12 site of Saccharomyces cerevisiae CICC1964;
more preferably, the modified yeast cell in (2) is obtained by integrating an aromatic aldehyde synthase derived from Petroselinum crispum into a delta12 site of Saccharomyces cerevisiae CICC1964, and substituting a fused chorismate mutase T/prephenate dehydrogenase derived from E. coli for a pdc1 gene of Saccharomyces cerevisiae CICC1964;
further preferably, the modified yeast cell in (2) is obtained by integrating an aromatic aldehyde synthase derived from Petroselinum crispum into a delta12 site of Kluyveromyces marxianus NBRC1777;
more preferably, the modified yeast cell is obtained by integrating an aromatic aldehyde synthase derived from Petroselinum crispum into a delta12 site of Kluyveromyces marxianus NBRC1777, and substituting a fused chorismate mutase T/prephenate dehydrogenase derived from E. coli for a pdc1 gene of Kluyveromyces marxianus NBRC1777.

7. The process according to claim 5, wherein the recombinant yeast for high tyrosol production is constructed.

8. The process according to claim 7, wherein the tyrosol is prepared by fermenting with the recombinant yeast;
preferably, the fermentation medium for fermentation contains at least one or a combination of two or more of glucose, fructose and sucrose, and tyrosine.

9. The process according to claim 7, wherein a hydroxytyrosol is prepared by fermenting with the recombinant yeast.

10. The process according to claim 9, wherein after the recombinant yeast for high tyrosol production is fermented to prepare tyrosol, the hydroxytyrosol is obtained through a 4-hydroxyphenylacetate hydroxylase reaction.

11. The process according to claim 10, wherein the tyrosol is catalyzed by E. coli of overexpressed 4-hydroxyphenylacetate hydroxylase to obtain the hydroxytyrosol.

12. The process according to claim 11, wherein the fermentation medium for fermentation contains at least one or a combination of two or more of glucose, fructose and sucrose, and tyrosine.
